(19) United States
(12) Patent Application Publication
(10) Pub. No.: US 2025/0120940 A1
(43) Pub. Date: Apr. 17, 2025

(54) COMPOSITION FOR IMPROVING FATIGUE, EXERCISE PERFORMANCE, OR ENDURANCE COMPRISING GALLOCATECHIN GALLATE AND ISOQUERCITRIN

(71) Applicant: AMOREPACIFIC CORPORATION, Seoul (KR)

(72) Inventors: Hyun Woo JEONG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/883,218

(22) Filed: Sep. 12, 2024

(30) Foreign Application Priority Data
Oct. 11, 2023 (KR) 10-2023-0135350

(51) Int. Cl. A61K 31/353; A61K 31/7048

(52) U.S. Cl. CPC A61K 31/353; A61K 31/7048

(57) ABSTRACT

A method for improving fatigue, exercise performance, or endurance, the method including administering an effective amount of Gallocatechin Gallate (GCG) and Isoquercitrin (IQC) to a subject in need thereof is disclosed. In an aspect, a composition including a complex component of Gallocatechin Gallate and Isoquercitrin can effectively inhibit accumulation of lactic acids in muscle cells and significantly increase mitochondria in the cells.

### CROSS REFERENCE TO RELATED APPLICATION

The present application claims the priority of Korean Patent Application No. 10-2023-0135350, filed on Oct. 11, 2023, the entire contents of which are hereby incorporated by this reference.

### BACKGROUND OF THE INVENTION

### Field of the Invention

The present specification discloses a composition for improving fatigue, exercise performance, or endurance.

### Description of the Related Art

The production of a large amount of ATP essentially requires oxidative phosphorylation reaction, which is energy metabolism through aerobic respiration in mitochondria. However, this oxidative phosphorylation reaction has a disadvantage of slow ATP production. In case the body's energy is rapidly depleted due to reasons such as exercise, if an amount of ATP produced is insufficient compared to the ATP consumed, ATP is quickly supplied through an anaerobic process instead of supplying a large amount of ATP through oxidative phosphorylation. A representative anaerobic ATP supply process is lactic acid fermentation. Glucose is produced into a pyruvic acid through glycolysis and then converted to acetyl coenzyme A, wherein the pyruvic acid is directly converted to a lactic acid by lactate dehydratase instead of being subjected to the citric acid cycle and electron transport system, and a small amount of ATP produced in this process is used as an energy source. Since the lactic acid that is accumulated during this process causes muscle fatigue and pain, it is recognized as a representative fatigue marker.

### SUMMARY OF THE INVENTION

A purpose of the present disclosure is to provide a composition for improving fatigue, exercise performance, or endurance.

In order to achieve the above purpose, in an aspect, the present disclosure provides a composition for improving fatigue, exercise performance or endurance, comprising Gallocatechin Gallate (GCG) and Isoquercitrin (IQC) as an active ingredient.

In an aspect, the composition of the present disclosure comprises a complex component of Gallocatechin Gallate and Isoquercitrin, which can effectively inhibit accumulation of lactic acids in muscle cells and significantly increase mitochondria in cells, thereby making it possible to improve exercise performance or alleviate fatigue by promoting energy production in situation that requires energy during exercise or in everyday life. The composition may be used as a material for foods or medicines as an exercise performance enhancer, an endurance enhancer, and a fatigue prevention and improvement agent.

### BRIEF DESCRIPTION OF THE DRAWINGS

FIG. 1 compares an effect of inhibiting expression of lactic acid fermentation-related genes in muscle cells according to a composition ratio of Gallocatechin Gallate (GCG) and Isoquercitrin (IQC) mixture (Adiphenon).

FIG. 2 compares an effect of inhibiting accumulation of lactic acids in muscle cells according to a composition ratio of Adiphenon.

FIG. 3 compares an effect of inhibiting accumulation of lactic acids in muscle cells according to a detailed composition ratio of Adiphenon.

FIG. 4 shows expression of mitochondria-related genes in muscle cells by Adiphenon.

FIG. 5 shows promotion of expression of fatty oxidation genes in muscle cells by Adiphenon.

FIG. 6 shows increase in mitochondria in muscle cells by Adiphenon.

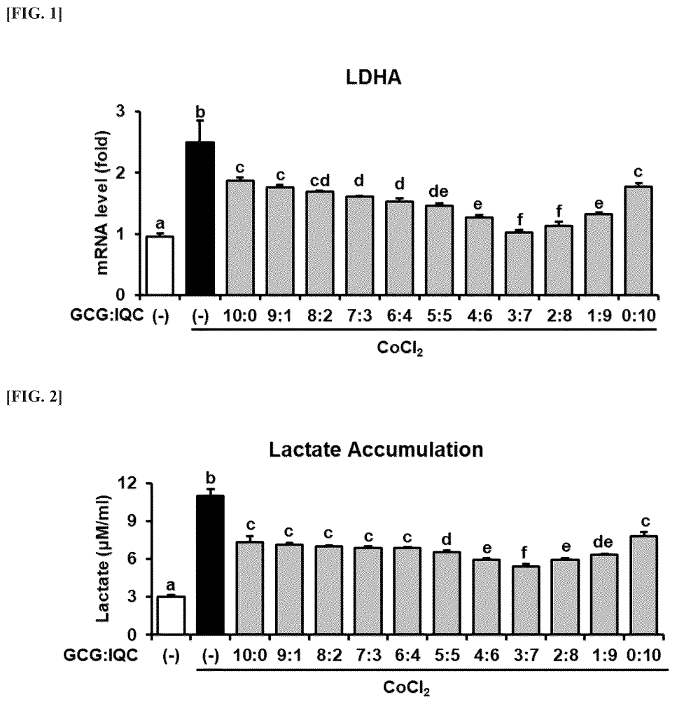

In FIGS. 1 to 6, experimental groups showing statistically significant differences (P<0.05) were indicated with different symbols.

### DETAILED DESCRIPTION OF THE INVENTION

Hereinafter, the present disclosure will be described in detail.

In an aspect, the present disclosure relates to a composition for improving fatigue, exercise performance, or endurance, the composition comprising Gallocatechin Gallate (GCG) and Isoquercitrin (IQC) as an active ingredient.

In an exemplary embodiment, Gallocatechin Gallate and Isoquercitrin may be contained in a weight ratio of 9:1 or more, 8:2 or more, 7:3 or more, 6:4 or more, 5:5 or more, or 4:6 or more, and may be contained in a weight ratio of 1:9 or less or 2:8 or less. For example, Gallocatechin Gallate and Isoquercitrin may be contained in a weight ratio of 9:1 to 1:9, but is not limited thereto.

In an exemplary embodiment, Gallocatechin Gallate and Isoquercitrin may be contained in a weight ratio of 1:1 or more, 1:1.1 or more, 1:1.2 or more, 1:1.3 or more, 1:1.4 or more, 1:1.5 or more, 1:1.6 or more, 1:1.7 or more, 1:1.8 or more, 1:1.9 or more, 1:2.0 or more, 1:2.1 or more, 1:2.2 or more, or 1:2.3 or more, and may be contained in a weight ratio of 1:9 or less, 1:8 or less, 1:7 or less, 1:6 or less, 1:5 or less, 1:4 or less, 1:3.5 or less, 1:3.4 or less, 1:3.3 or less, 1:3.2 or less, 1:3.1 or less, 1:3 or less, 1:2.9 or less, 1:2.8 or less, 1:2.7 or less, 1:2.6 or less, or 1:2.5 or less. In case Gallocatechin Gallate and Isoquercitrin are contained in the above weight ratio, fatigue, exercise performance, or endurance can be effectively improved. For example, Gallocatechin Gallate and the Isoquercitrin may be contained in a weight ratio of 1:1 to 1:9 or 1:1.5 to 1:4, but is not limited thereto.

In an exemplary embodiment, the composition may be characterized in that it is administered to a subject that needs to inhibit accumulation of lactic acids in muscle cells.

In an exemplary embodiment, the composition may be characterized in that it is administered to a subject that needs to reduce expression of LDHA (Lactate dehydrogenase A) genes.

In an exemplary embodiment, the composition may be characterized in that it is administered to a subject that needs to increase aerobic energy metabolism.

In an exemplary embodiment, the composition may be characterized in that it is administered to a subject that needs to increase expression of mitochondria or fatty oxidation-related genes, wherein the mitochondria-related genes may be a NDUFA9 (NADH: ubiquinone oxidoreductase subunit A9) gene or a COX4 (Cytochrome C Oxidase Subunit 4) gene, and the fatty oxidation-related genes may be an ACO (Acyl-CoA Oxidase) gene or a CPT-1 (Carnitine-Palmitoyl Transferase 1) gene.

In an exemplary embodiment, the composition may be characterized in that it is administered to a subject that needs to increase mitochondria in muscle cells.

In an exemplary embodiment, a daily dosage of the active ingredient in the composition may be 5 mg/kg or more, 6 mg/kg or more, 7 mg/kg or more, 8 mg/kg or more, 9 mg/kg or more, 10 mg/kg or more, 20 mg/kg or more, 30 mg/kg or more, 40 mg/kg or more, 50 mg/kg or more, 60 mg/kg or more, 70 mg/kg or more, 80 mg/kg or more, 90 mg/kg or more, 100 mg or more, 1 g/kg or more, 2 g/kg or more, 3 g/kg or more, 4 g/kg or more, 5 g/kg or more, or 6 g/kg or more, and may be 60 g/kg or less, 59 g/kg or less, 58 g/kg or less, 57 g/kg or less, 56 g/kg or less, 55 g/kg or less, 54 g/kg or less, 53 g/kg or less, 52 g/kg or less, or 51 g/kg or less. An effect of improving fatigue, exercise performance, or endurance is excellent at the above dosage. If the dosage is less than the above range, the effect of improving fatigue, exercise performance, or endurance is minimal, and if the dosage is higher than the above range, a problem such as toxicity may occur. The above dosage may be administered once or in divided doses several times per a day. For example, it may be administered 2 to 24 times per a day, 1 to 2 times per 3 days, 1 to 6 times per a week, 1 to 10 times per 2 weeks, 1 to 15 times per 3 weeks, 1 to 3 times per 4 weeks, or 1 to 12 times per a year, but is not limited thereto.

In an exemplary embodiment, the composition may be a food composition.

In an exemplary embodiment, in case the composition is used as an additive for a health functional food, the composition may be added as it is or used together with other foods or food ingredients, and may be used appropriately according to conventional methods. A mixed amount of the active ingredients may be appropriately determined depending on each purpose of use, such as prevention, health, or treatment. A formulation of the health functional food may be in any form of powders, granules, pills, tablets or capsules as well as general foods or beverages.

In an exemplary embodiment, there is no particular limitation on the type of the health functional food, and examples of foods to which the composition can be added include meat, confectionery, noodles, gum, dairy products including ice cream, various soups, beverages, tea, drinks, alcoholic beverages, vitamin complexes, etc., and may include all foods in the conventional sense.

In an exemplary embodiment, in the preparation of the health functional food or beverage, the composition may be added in an amount of 15 parts by weight or less, preferably 10 parts by weight or less, based on 100 parts by weight of the raw material. However, in the case of long-term intake for the purpose of health and hygiene or health control, the above amount may be below said range.

In an exemplary embodiment, the beverages among the health functional food may contain various flavoring agents or natural carbohydrates as additional ingredients like the typical beverages. The above-mentioned natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. A sweetening agent may include natural sweetening agents such as thaumatin and stevia extract or synthetic sweetening agents such as saccharin and aspartame. A ratio of the natural carbohydrates may be about 0.01 to 0.04 g, preferably about 0.02 to 0.03 g, based on 100 mL of the beverages according to the present disclosure, but are not limited thereto.

In an exemplary embodiment, in addition to the above, the health functional food according to the present disclosure may contain various nutrients, vitamins, electrolytes, flavors, colorants, pectic acids and their salts, alginic acids and their salts, organic acids, protective colloidal thickeners, pH regulators, stabilizers, preservatives, glycerin, alcohol, and carbonating agents used in carbonated beverages. In addition, the health functional food according to the present disclosure may contain fruit flesh for the production of natural fruit juice, fruit juice drinks, and vegetable drinks. These ingredients can be used independently or in combination with each other. A ratio of these additives is not limited, but is generally selected in the range of 0.01 to 0.1 parts by weight based on 100 parts by weight of the health functional food according to the present disclosure.

In other aspect, the present disclosure provides a method for improving fatigue, exercise performance, or endurance, the method comprising administering to a subject a composition comprising an effective amount of Gallocatechin Gallate (GCG) and Isoquercitrin (IQC).

In another aspect, the present disclosure provides a use of Gallocatechin Gallate (GCG) and Isoquercitrin (IQC) for preparing the composition for improving fatigue, exercise performance, or endurance.

In still another aspect, the present disclosure provides Gallocatechin Gallate (GCG) and Isoquercitrin (IQC) for improving fatigue, exercise performance, or endurance.

In more still another aspect, the present disclosure provides a non-therapeutic use of Gallocatechin Gallate (GCG) and Isoquercitrin (IQC) for improving fatigue, exercise performance, or endurance.

Hereinafter, the constitutions and effects of the present disclosure will be described in more detail through Examples. However, since the following Examples are provided only for illustrative purposes to aid understanding of the present disclosure, the scope and range of the present disclosure should not be limited by them.

### EXAMPLE

### Example 1

### Comparison of an Effect of Inhibiting Accumulation of Fatigue Substances in Muscle Cells by Treatment with a Mixture (Adiphenon) of Gallocatechin Gallate (GCG) and Isoquercitrin (IQC)

In order to check whether accumulation of lactic acids in muscle cells is inhibited, mouse-derived myofibroblast C2C12 cells were purchased from ATCC and cultured using Dulbecco's Modified Eagle's Medium (DMEM; Sigma Aldrich) containing 10% fetal bovine serum (Gibco) and 1% penicillin/streptomycin (P/S; Sigma Aldrich). When the C2C12 cells were 100% full in a culture dish, they were differentiated into the muscle cells using a DMEM medium containing 2% horse serum (Gibco) and 1% P/S. Differentiation into the muscle cells took about 7 days, and the medium was changed every day during differentiation of the muscle cells.

When the cells are treated with CoCl₂, they are recognized as hypoxia despite the presence of oxygen and initiate fermentation of lactic acids. Accordingly, CoCl₂ can be used to induce accumulation of the lactic acids in the cells. In order to check whether a mixture (Adiphenon) of Gallocatechin Gallate (GCG) and Isoquercitrin (IQC) can inhibit accumulation of the lactic acids in the muscle cells, the differentiated muscle cells were pretreated with various ratios of Adiphenon (GCG:IQC).=10:0˜0:10) for 24 hours and then treated with CoCl₂ (100 μM; Sigma Aldrich). Thereafter, the medium was collected and used to measure an amount of the accumulated lactic acids, and the cells were washed with a phosphate buffered saline (PBS) and used for experiments of measuring RNA expression. RNA was extracted from the collected cells using TaKaRa MiniBEST Universal RNA Extraction Kit (Takara Bio), and then cDNA was synthesized from the same amount (1 mg) of RNA using RevertAid 1^{st}-strand cDNA Synthesis Kit (Thermo Fisher Scientific). Expression of lactic acid fermentation-related genes (lactate dehydrogenase A) was observed using a CFX96 thermocycler (Bio-Rad), and an amount of accumulated lactic acids was quantified using a Lactate Assay Kit (Sigma Aldrich).

As a result, as shown in FIGS. 1 and 2, an effect of inhibiting fermentation of the lactic acids in the muscle cells was confirmed by Adiphenon of various composition ratios, and the effect was found to be best at a ratio of GCG:IQC=4:6 to 2:8.

### Example 2

### Comparison of an Effect According to Ratios of Adiphenon

The results of Example 1 showed that Adiphenon inhibited accumulation of lactic acids by CoCl₂ treatment, and that the effect of inhibiting accumulation of the lactic acids varied depending on composition ratios of Adiphenon. In order to check an optimal composition ratio of GCG and IQC constituting Adiphenon, the ratio of GCG:IQC (4:6˜2:8), which showed the best effect in inhibiting accumulation of the lactic acids, was further subdivided to compare an effect of inhibiting accumulation of the lactic acids.

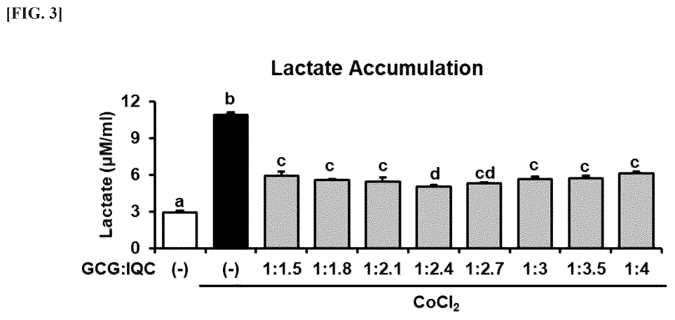

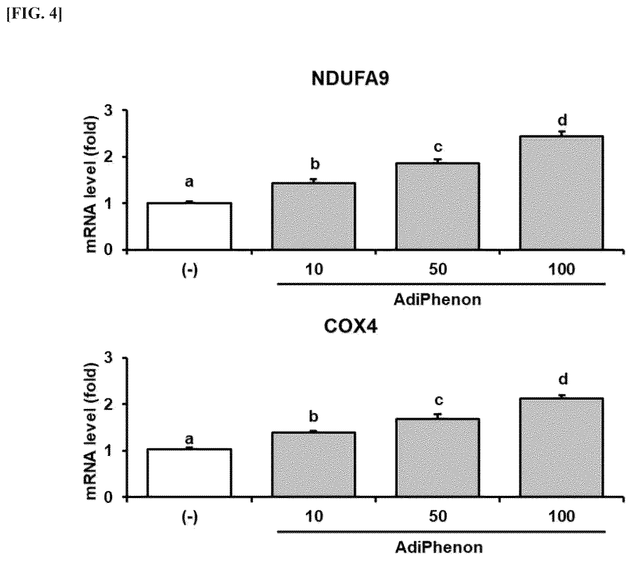

As a result, as shown in FIG. 3, it was found that the effect of inhibiting accumulation of the lactic acids was the best when the ratio of GCG:IQC was 1:2.4. Accordingly, in subsequent Examples, experiments were performed with the composition ratio of Adiphenon having GCG:IQC=1:2.4.

### Example 3

### Comparison of Energy Metabolism Activation in Muscle Cells by Adiphenon Treatment

In order to check whether the effect of inhibiting accumulation of the lactic acids by Adiphenon is due to increase in aerobic energy metabolism, the differentiated muscle cells were treated with Adiphenon at various concentrations (10, 50, 100 μg/ml) for 24 hours, and then expression of the mitochondria and fatty oxidation-related genes was confirmed.

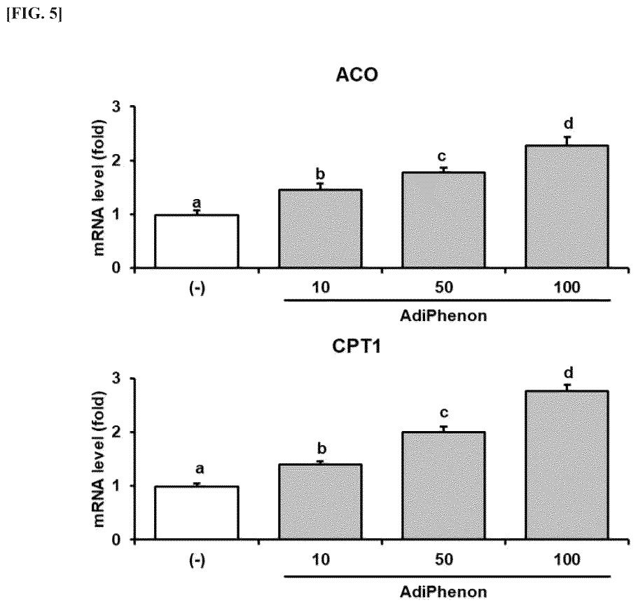

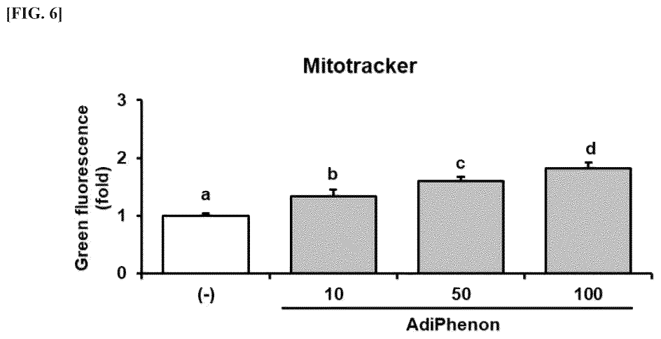

As a result, as shown in FIGS. 4 and 5, expression of the mitochondrial components and fatty oxidation-related genes was increased in a concentration-dependent manner due to Adiphenon treatment.

Next, in order to find out whether the mitochondria actually were increased in the muscle cells due to Adiphenon treatment, after Adiphenon was treated under the same conditions, the cells were washed with PBS, and the mitochondria were stained by treatment with Mitotracker™ Green FM (20 nM; Invitrogen) for 30 minutes. Thereafter, the stained cells were washed twice more with PBS to remove the remaining Mitotracker, and then fluorescence values (ex 490 nm, em 512 nm) were measured using a TECAN Infinite M200 Microplate Reader.

As a result, as shown in FIG. 6, it was confirmed that an amount of the mitochondria in the muscle cells was increased by Adiphenon treatment, which is the result consistent with increase in expression of the mitochondria-related genes by Adiphenon treatment in FIG. 4.

What is claimed is:

1. A method for improving fatigue, exercise performance or endurance, the method comprising administering an effective amount of Gallocatechin Gallate (GCG) and Isoquercitrin (IQC) to a subject in need thereof.

2. The method according to claim 1, wherein Gallocatechin Gallate (GCG) and Isoquercitrin (IQC) are contained in a weight ratio of 9:1 to 1:9.

3. The method according to claim 1, wherein the subject needs to inhibit accumulation of lactic acids in muscle cells.

4. The method according to claim 1, wherein the subject needs to increase aerobic energy metabolism.

5. The method according to claim 1, wherein the subject needs to increase expression of mitochondria or fatty oxidation-related genes.

6. The method according to claim 1, wherein the subject needs to increase mitochondria in muscle cells.
